(19) United States
(12) Patent Application Publication
(10) Pub. No.: US 2025/0268758 A1
(43) Pub. Date: Aug. 28, 2025

(54) GOGGLE LENS REPLACEMENT STRUCTURE

(71) Applicant: PROHERO GROUP CO., LTD., TAINAN CITY (TW)

(72) Inventor: PEN-WEI CHEN, TAINAN CITY (TW)

(21) Appl. No.: 18/587,000

(22) Filed: Feb. 26, 2024

(51) Int. Cl. A61F 9/02

(52) U.S. Cl. CPC A61F 9/025

(57) ABSTRACT

The invention relates to a goggle lens replacement structure. The lens assembly hole includes a limiting convex portion protruding on one side, and an embedding convex portion protruding on the other side of the spectacle frame. The spectacle lens is arranged in the lens assembly hole. The spectacle lens includes an embedded hole provided on one side corresponding to the limiting convex portion, and a fitting portion arranged on the other side thereof. The positioning device is combined and fixed with the fitting portion. The positioning device has a slotted hole disposed thereon corresponding to the embedding convex portion, and a movable element movable in the slotted hole, so that the movable element can be pushed by a user to move in the slotted hole. The movable element has an embedding protrusion protrudes formed a side thereof toward the outside of the slotted hole, corresponding to the embedding convex portion.

### BACKGROUND OF THE PRESENT INVENTION

### Field of Invention

The present invention relates to a goggle replacement structure, in particular to the goggle lens replacement structure that makes the assembly and positioning of the spectacle lens more simple and convenient, and when the spectacle lens is to be replaced, the spectacle lens can be easily and conveniently separated from the lens assembly hole, so that the spectacle lens can be easily removed and replaced, so that it can also be more simple and convenient in the disassembly of the spectacle lens, and the goggle replacement structure innovation design that adds practical functional characteristics in its overall implementation and use.

### Description of Related Arts

When people engage in various outdoor activities, such as mountain climbing, skiing, etc., or when people engage in other high-speed activities, such as skydiving, riding high-speed motorcycles, etc., people often wear goggles on their faces to use the goggles to shield their eyes to prevent their eyes from wind and snow and causing blurred vision, and at the same time to avoid eye injuries, thus achieving the effect of protecting their eyes.

In terms of the structural design of this type of goggles, which generally have a penetrated hollow lens assembly hole formed on the front end of a frame, and a lens mounting groove is formed around the lens assembly hole, so that the lens can be assembled in the lens assembly hole, and the periphery of the lens is combined and fixed with the lens mounting groove. When the goggles are worn by the user, the frame of the goggles can fit on the user's face around the eyes, so that the lens provided in the lens assembly hole can be used to shield the user's eyes to achieve the effect of allowing users to clearly see the surrounding environment while at the same time protecting the user's eyes.

However, although the above-mentioned goggles can achieve the expected effect of being worn by users to protect their eyes, it is also found in their actual operation and use that if the lenses of such goggles are worn, scratched or cracked and need to be disassembled and replaced, they not only need to be pulled open by both sides of the frame, so that the lens and the lens mounting groove are separated from a combined state, and usually need to be disassembled with the help of other tools, which is extremely labor-intensive and time-consuming, and it is also extremely inconvenient when the new lens is to be assembled. As a result, there is still room for improvement in the overall structural design.

In view of this, the inventor adheres to many years of rich experience in design, development and practical production in the related industry, researches and improves the conventional structure and defects, and provides a goggle replacement lens structure, in order to achieve the purpose of better practical value.

### SUMMARY OF THE PRESENT INVENTION

The main purpose of the present invention is to provide a goggle lens replacement structure, which mainly makes the assembly and positioning of the lenses simpler and more convenient. When the spectacle lens is to be replaced, the movable element of the positioning device can be pushed to easily separate the spectacle lens from the combined state with the lens assembly hole of the spectacle frame making it easier to separate the spectacle lens from the lens assembly hole and easily remove the spectacle lens for replacement. The disassembly of the spectacle lens can also be made easier and more convenient, and its overall implementation and use have more practical features.

The main purpose and effect of the goggle lens replacement structure of the present invention are achieved by the following specific technical means:
the goggle lens replacement structure includes a spectacle frame, a spectacle lens, and a positioning device, wherein
the spectacle frame comprises a through-hollow lens assembly hole formed at a front end thereof, wherein the lens assembly hole comprises a limiting convex portion protruding on one side, and an embedding convex portion protruding on the other side;
the spectacle lens is arranged corresponding to the lens assembly hole of the spectacle frame, so that the spectacle lens is capable of being installed in the lens assembly hole, wherein the spectacle lens comprises an embedded hole provided on one side thereof corresponding to the limiting convex portion of the lens assembly hole, and a fitting portion arranged on the other side of the spectacle lens;
the positioning device is combined and fixed with the fitting portion of the spectacle lens, wherein the positioning device has a slotted hole disposed thereon corresponding to the embedding convex portion of the lens assembly hole of the spectacle frame, and a movable element movable in the slotted hole, so that the movable element is capable of being pushed by a user to move in the slotted hole, wherein the movable element has an embedding protrusion protrudes formed a side thereof toward the outside of the slotted hole, corresponding to the embedding convex portion, and capable of being engaged with each other.

The preferred embodiment of the goggle lens replacement structure of the present invention, the spectacle frame comprises a coupling convex portion protruding from a middle upper end edge corresponding to the lens assembly hole, wherein the spectacle lens comprises a concave portion formed at an upper edge thereof corresponding to the coupling convex portion, two corresponding coupling holes provided on both sides of the concave portion, and a decorative block fixed in combination with the coupling holes, wherein the decorative block is capable of being engaged and combined with the coupling convex portion through the concave portion.

The preferred embodiment of the goggle lens replacement structure of the present invention, the spectacle frame includes a resisting portion formed at a middle lower end edge corresponding to the lens assembly hole, so that the spectacle lens abuts a front end of the resisting portion.

The preferred embodiment of the goggle lens replacement structure of the present invention, the spectacle frame includes two corresponding inlay convex portions arranged below two sides of the middle of the lens assembly hole, wherein the spectacle lens includes a positioning insertion hole provided at a middle lower end thereof, and a nose bridge block installed thereon, wherein the nose bridge block has a positioning insertion part inserted and positioned in combination with the positioning insertion hole, and two embedded recessed portions recessed on two sides thereof, so as to be fixed and embedded in position corresponding to the inlay convex portions of the spectacle frame.

### BRIEF DESCRIPTION OF THE DRAWINGS

FIG. 1 is an exploded view according to the present invention.

FIG. 2 is a partially exploded view according to the present invention.

FIG. 3 is an assembly perspective view according to the present invention.

FIG. 4 is a side assembly sectional view according to the present invention.

FIG. 5 is a top assembly sectional view according to the present invention.

FIG. 6 is a perspective view (1) of the lens in a disassembled state according to the present invention.

FIG. 7 is a perspective view (2) of the lens in a disassembled state according to the present invention.

### DETAILED DESCRIPTION OF THE PREFERRED EMBODIMENT

In order to have a more complete and clear disclosure of the technical content, the purpose of invention and the effect achieved by the present invention, it is explained in detail below, and refer to the disclosed drawings and drawing numbers together.

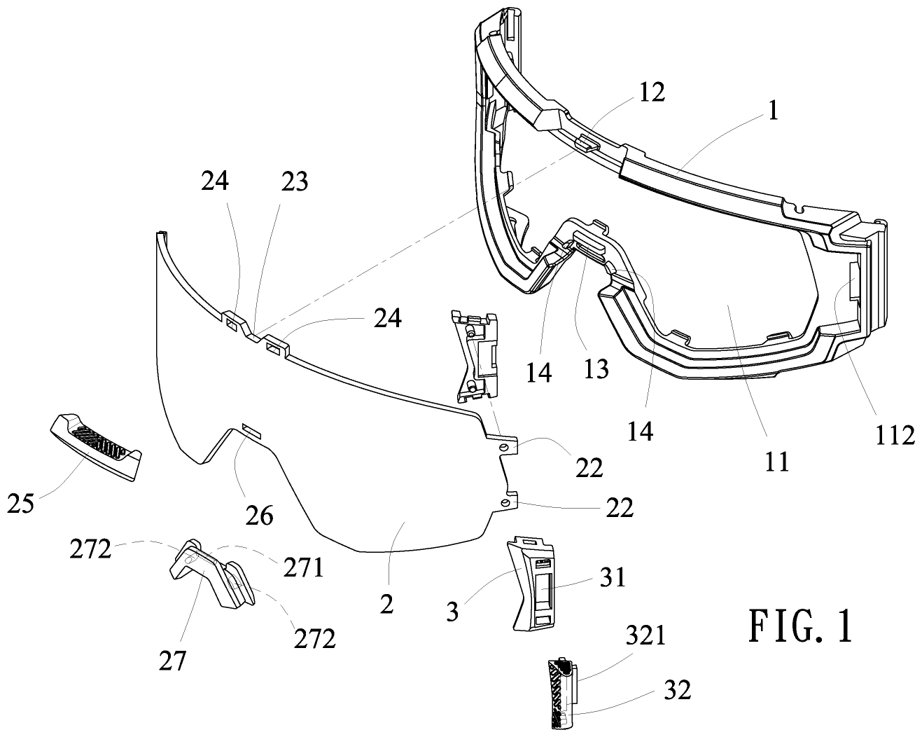

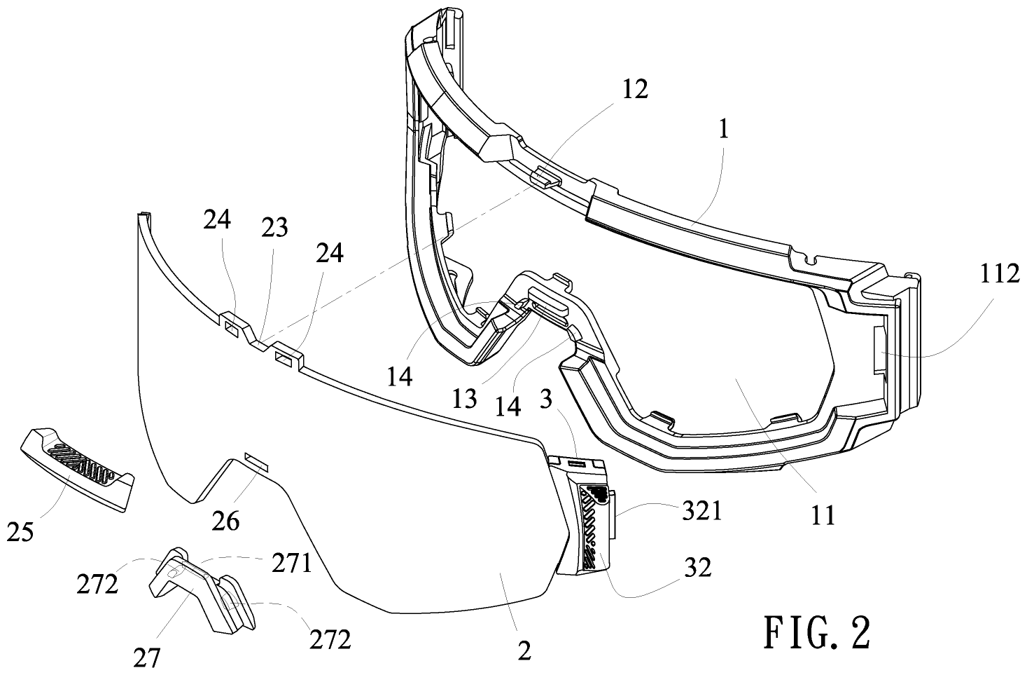

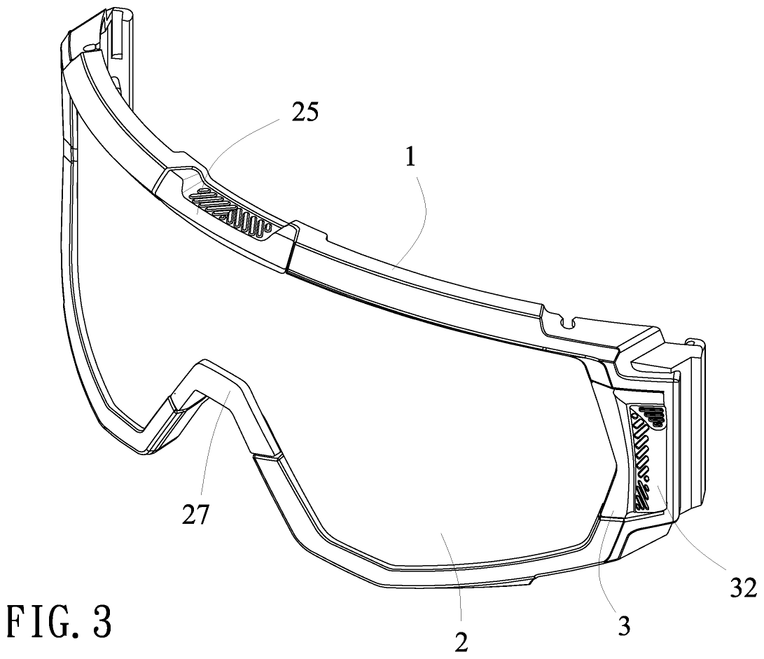

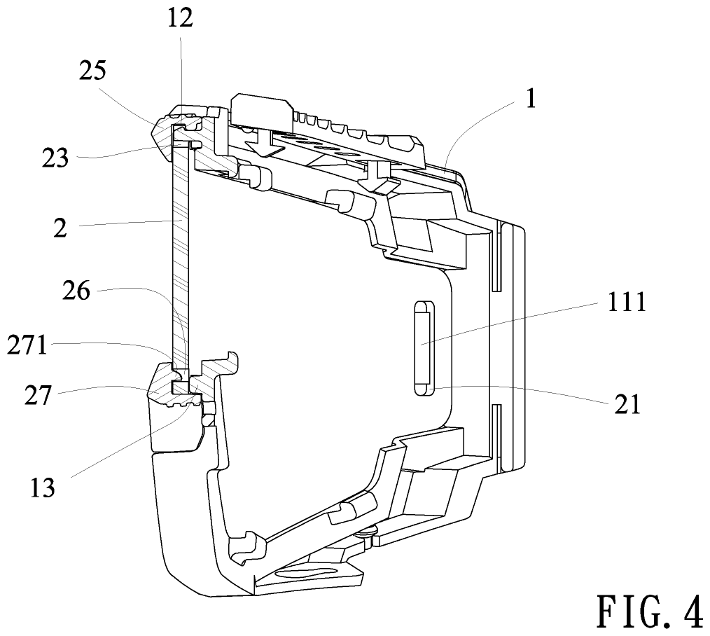

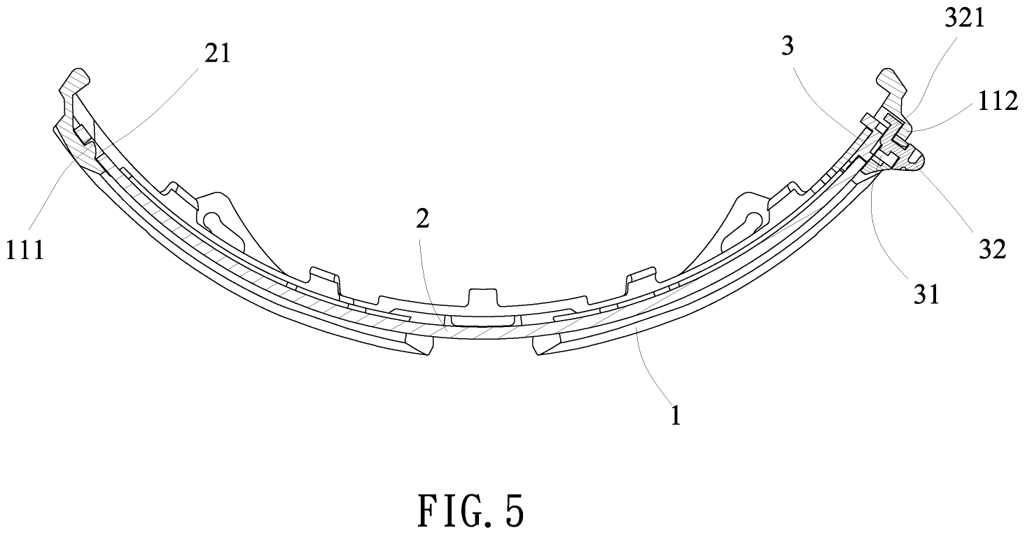

First, referring to the exploded view of the present invention in FIG. 1, the partial exploded view of the present invention in FIG. 2, the assembly perspective view of the present invention in FIG. 3, the side assembly sectional view of the present invention in FIG. 4 and the top assembly sectional view of the present invention in FIG. 5, the present invention mainly includes a spectacle frame (1), a spectacle lens (2), and a positioning device (3), wherein

the spectacle frame (1) includes a through-hollow lens assembly hole (11) formed at the front end thereof, a coupling convex portion (12) protruding from a middle upper end edge corresponding to the lens assembly hole (11), a resisting portion (13) formed at a middle lower end edge corresponding to the lens assembly hole (11), and two corresponding inlay convex portions (14) respectively arranged on two sides of the resisting portion (13), wherein the lens assembly hole (11) includes a limiting convex portion (111) protruding on one side, and an embedding convex portion (112) protruding on the other side.

The spectacle lens (2) is arranged corresponding to the lens assembly hole (11) of the spectacle frame (1), so that the spectacle lens (2) is capable of being installed in the lens assembly hole (11), wherein the spectacle lens (2) includes an embedded hole (21) provided on one side thereof corresponding to the limiting convex portion (111) of the lens assembly hole (11), a fitting portion (22) arranged on the other side of the spectacle lens (2), a concave portion (23) formed at the upper edge of the spectacle lens (2) corresponding to the coupling convex portion (12) of the spectacle frame (1), two corresponding coupling holes (24) provided on both sides of the concave portion (23), a decorative block (25) fixed in combination with the coupling holes (24), which is capable of being engaged and combined with the coupling convex portion (12) through the concave portion (23), a positioning insertion hole (26) provided at the middle lower end of the spectacle lens (2), and a nose bridge block (27) installed thereon, wherein the nose bridge block (27) has a positioning insertion part (271) inserted and positioned in combination with the positioning insertion hole (26), and two embedded recessed portions (272) recessed on both sides thereof, so as to be fixed and embedded in position corresponding to the inlay convex portions (14) of the spectacle frame (1).

The positioning device (3) is used to be combined and fixed with the fitting portion (22) of the spectacle lens (2), wherein the positioning device (3) has a slotted hole (31) disposed thereon corresponding to the embedding convex portion (112) of the lens assembly hole (11) of the spectacle frame (1), and the movable element (32) movable in the slotted hole (31), so that the movable element (32) can be pushed by the user to move in the slotted hole (31), wherein the movable element (32) has an embedding protrusion (321) protruding from the side thereof toward the outside of the slotted hole (31) corresponding to the embedding convex portion (112).

In this way, when the present invention is assembled and combined, the spectacle lens (2) is arranged in the lens assembly hole (11) of the spectacle frame (1), the embedded hole (21) on one side of the spectacle lens (2) is sleeved on the limiting convex portion (111) of the lens assembly hole (11), the positioning device (3) and the embedding convex portion (112) of the lens assembly hole (11) are combined on the other side of the spectacle lens (2), so that the movable element (32) of the positioning device (3) is positioned by the embedding protrusion (321) and the embedding convex portion (112), which are embedded and combined, that is, the spectacle lens (2) is indeed assembled in the lens assembly hole (11) and does not fall off. At this time, the spectacle lens (2) abuts the front end of the resisting portion (13), the coupling convex portion (12) of the spectacle frame (1) passes through the concave portion (23) and is engaged and fixed with the decorative block (25) fixed at the coupling holes (24) of the spectacle lens (2), the positioning insertion part (271) of the nose bridge block (27) and the positioning insertion hole (26) of the spectacle lens (2) are inserted into each other and positioned together, so that the two sides of the nose bridge block (27) are fixedly embedded and positioned by the embedded recessed portions (272) thereof and the inlay convex portions (14) of the spectacle frame (1).

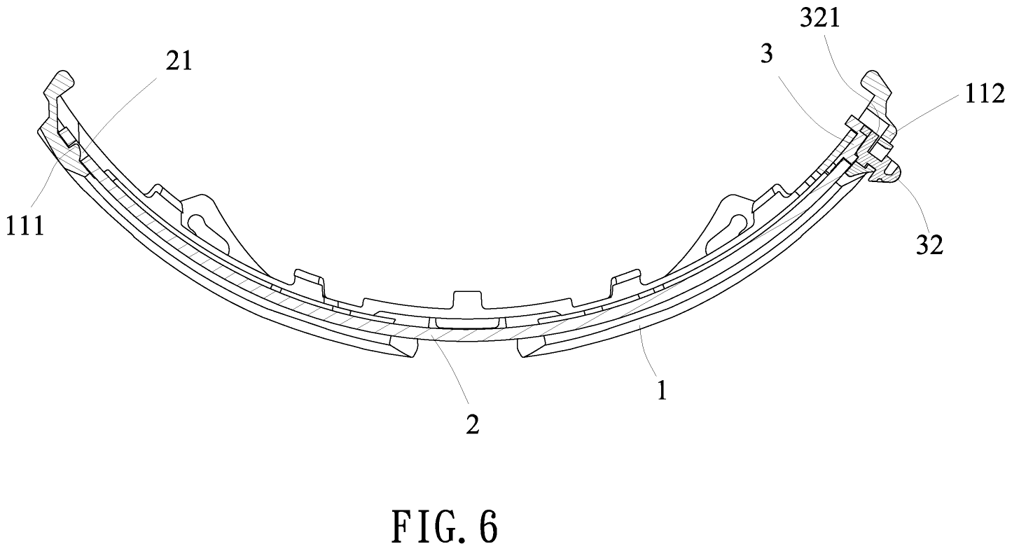

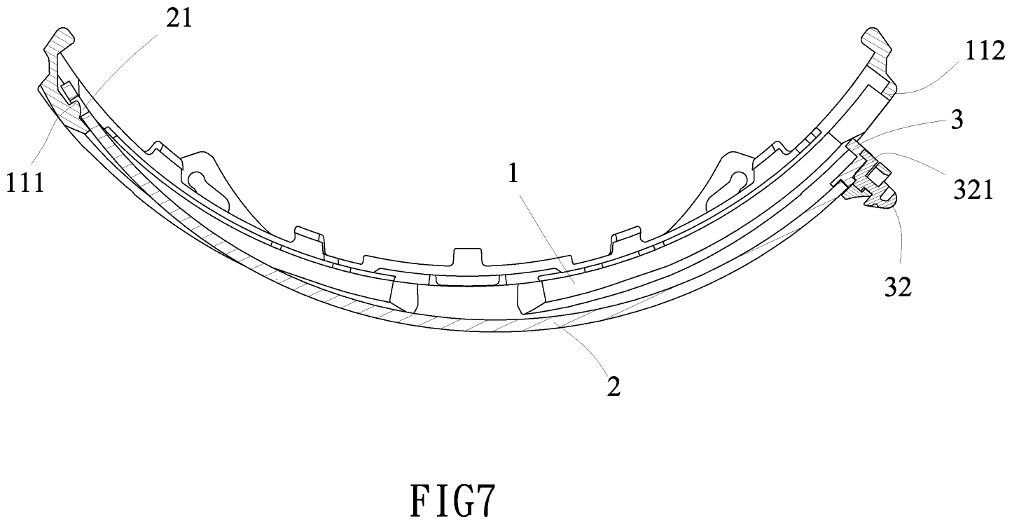

When the spectacle lens (2) is to be replaced, the movable element (32) of the positioning device (3) is pushed in the opposite direction of the embedding convex portion (112) of the lens assembly hole (11), so that the embedding protrusion (321) and the embedding convex portion (112) are separated from the combined state [refer to the spectacle lens disassembly state view (1) of the present invention in FIG. 6], that is, the side of the spectacle lens (2) provided with the positioning device (3) can be separated from the lens assembly hole (11), [refer to the spectacle lens disassembly state view (2) of the present invention in FIG. 7], and relatively detached the embedded hole (21) of the spectacle lens (2) from the limiting convex portion (111) of the lens assembly hole (11) to remove the spectacle lens (2). When the spectacle lens (2) is to be assembled, the embedded hole (21) of the spectacle lens (2) is first combined with the limiting convex portion (111) of the lens assembly hole (11), and the spectacle lens (2) is pushed into the lens assembly hole (11), and then the movable element (32) of the positioning device (3) is pushed in the direction of the embedding convex portion (112) of the lens assembly hole (11), so that the embedding protrusion (321) of the movable element (32) is positioned in combination with the embedding convex portion (112), that is, the spectacle lens (2) can indeed be fixed in combination with the lens assembly hole (11).

It can be seen from the above description of the use of the present invention that compared with the conventional technical means, the present invention mainly makes the assembly and positioning of the spectacle lens simpler and more convenient. When the spectacle lens is to be replaced, the movable element of the positioning device can be pushed to easily separate the spectacle lens from the combined state with the lens assembly hole of the spectacle frame, making it easier to separate the spectacle lens from the lens assembly hole and easily remove the spectacle lens for replacement. The disassembly of the spectacle lens can also be made easier and more convenient, and its overall implementation and use have more practical features.

What is claimed is:

1. A goggle lens replacement structure, comprising a spectacle frame, a spectacle lens, and a positioning device, wherein
the spectacle frame comprises a through-hollow lens assembly hole formed at a front end thereof, wherein the lens assembly hole comprises a limiting convex portion protruding on one side, and an embedding convex portion protruding on the other side;
the spectacle lens is arranged corresponding to the lens assembly hole of the spectacle frame, so that the spectacle lens is capable of being installed in the lens assembly hole, wherein the spectacle lens comprises an embedded hole provided on one side thereof corresponding to the limiting convex portion of the lens assembly hole, and a fitting portion arranged on the other side of the spectacle lens;
the positioning device is combined and fixed with the fitting portion of the spectacle lens, wherein the positioning device has a slotted hole disposed thereon corresponding to the embedding convex portion of the lens assembly hole of the spectacle frame, and a movable element movable in the slotted hole, so that the movable element is capable of being pushed by a user to move in the slotted hole, wherein the movable element has an embedding protrusion protrudes formed a side thereof toward the outside of the slotted hole, corresponding to the embedding convex portion, and capable of being engaged with each other.

2. The goggle lens replacement structure as claimed in claim 1, wherein the spectacle frame comprises a coupling convex portion protruding from a middle upper end edge corresponding to the lens assembly hole, wherein the spectacle lens comprises a concave portion formed at an upper edge thereof corresponding to the coupling convex portion, two corresponding coupling holes provided on both sides of the concave portion, and a decorative block fixed in combination with the coupling holes, wherein the decorative block is capable of being engaged and combined with the coupling convex portion through the concave portion.

3. The goggle lens replacement structure as claimed in claim 1, wherein the spectacle frame comprises two corresponding inlay convex portions arranged below two sides of the middle of the lens assembly hole, wherein the spectacle lens comprises a positioning insertion hole provided at a middle lower end thereof, and a nose bridge block installed thereon, wherein the nose bridge block has a positioning insertion part inserted and positioned in combination with the positioning insertion hole, and two embedded recessed portions recessed on two sides thereof, so as to be fixed and embedded in position corresponding to the inlay convex portions of the spectacle frame.

4. The goggle lens replacement structure as claimed in claim 1, wherein the spectacle frame comprises a resisting portion formed at a middle lower end edge corresponding to the lens assembly hole, so that the spectacle lens abuts a front end of the resisting portion.

5. The goggle lens replacement structure as claimed in claim 4, wherein the spectacle frame comprises two corresponding inlay convex portions arranged below two sides of the middle of the lens assembly hole, wherein the spectacle lens comprises a positioning insertion hole provided at a middle lower end thereof, and a nose bridge block installed thereon, wherein the nose bridge block has a positioning insertion part inserted and positioned in combination with the positioning insertion hole, and two embedded recessed portions recessed on two sides thereof, so as to be fixed and embedded in position corresponding to the inlay convex portions of the spectacle frame.
